(19) United States
(12) Patent Application Publication
(10) Pub. No.: US 2010/0063387 A1
(43) Pub. Date: Mar. 11, 2010

(54) POINTING DEVICE FOR MEDICAL IMAGING

(75) Inventor: Holger Timinger, Hamburg (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/527,946

(22) PCT Filed: Feb. 22, 2008
(86) PCT No.: PCT/IB2008/050653

§ 371 Date: Aug. 20, 2009

(30) Foreign Application Priority Data
Feb. 26, 2007 (EP) 07103078.7

(51) Int. Cl. A61B 5/05

(52) U.S. Cl. 600/426

(57) ABSTRACT

The invention relates to a pointing device (40) for indicating the spatial position of markers (2) to a localization system (10). The pointing device (40) comprises a sensor (42) that detects if a definite interaction with the marker (2) takes place. The sensor (42) may for example be a pressure sensor (42) that determines if the contact force exceeds a threshold which could lead to undesired shifts of the marker (2). The pointing device (40) is used to determine the spatial coordinates of the marker (2), which can then be registered with the image coordinates of the marker (2) in a stored image, e.g. a CT-image.

### FIELD OF THE INVENTION

The invention relates to a pointing device for indicating the spatial position of a marker to a tracking device. Moreover, it relates to a localization system and an investigation apparatus comprising such a pointing device.

### BACKGROUND OF THE INVENTION

For animal experiments a setup has been described in literature (cf. J. C. Li, I. Iordachita, E. Balogh, G. Fichtinger and P. Kazanzides: “Validation of an Image-Guided Robot System for Measurement, Biopsy and Injection in Rodents”, Bioengineering Conference, 2005, Proceedings of the IEEE 31st Annual Northeast, pp. 131-133) that uses pre-operatively generated images of a rodent and a robot system for introducing a needle into target regions of a tumor identified on the images. A registration between the image coordinates and robot coordinates is achieved by guiding the robot manually to fiducial markers in the animal body.

### SUMMARY OF THE INVENTION

Based on this background it was an object of the present invention to provide means for the determination of the spatial position of markers that can readily be used in different medical procedures and provide a high accuracy of localization.

This object is achieved by a pointing device according to claim 1, a localization system according to claim 2, and an investigation apparatus according to claim 3. Preferred embodiments are disclosed the dependent claims.

According to its first aspect, the invention relates to a pointing device for indicating the spatial position of at least one marker to an associated tracking device. The pointing device will usually be a comparatively light, manually held, pencil-like object. It is equipped with the means that are needed for localizing it with a tracking device, for example with a coil or magnet in a magnetic tracking environment or generally a source, reflector or sensor for some physical quantity like light or sound. The pointing device comprises the following
a) A tip that can be located by the tracking device and that can be brought into a definite interaction with the marker. The term “tip” is to be understood in this context in a broad sense just as a name for some dedicated region of the pointing device; the name indicates however that said region is typically (but not necessarily) an exposed, tapered part of the pointing device. The localization of said tip by the tracking device can be achieved directly or indirectly; in the latter case, the tracking device actually determines the position of one or more different points on the pointing device, from which the position of the tip can be inferred (e.g. because both are located on the same solid body).
   As will be explained in more detail below, the “definite interaction” can for example be a physical contact between tip and marker. Moreover, it may comprise a desired state that should prevail during a measurement and/or an undesirable state that should be avoided during a measurement.
b) A sensor for detecting if the mentioned definite interaction between tip and marker takes place. In continuation of the given example, the sensor may for example determine if the tip and the marker are in physical contact.
   It should be noted that the sensor needs not necessarily be coupled to the tip of the pointing device. It may even be completely separate from the body carrying the tip and for example be fixed to the marker (the localization of a plurality of markers with one pointing device would then require a plurality of sensors or a transfer of the sensor from marker to marker). Preferably, both the tip and the sensor are however mechanically coupled to each other.

The equipment of the described pointing device with the sensor has the advantage that a user has an objective control if the pointing device is in its prescribed position with respect to the marker for correctly indicating the spatial position of the marker to the associated tracking device. If no such sensor would be present, it would be left to the user to determine e.g. by visual inspection or haptically if the pointing device is correctly positioned with respect to the considered marker. A contact with too much pressure could under these circumstances shift the marker and thus lead to erroneous position measurements which would have to be repeated or would affect the accuracy of the whole procedure. In contrast to this, the sensor of the proposed pointing device can objectively indicate a “good” and/or a “bad” positioning for the measurements. Moreover, the detection of a “good” positioning can be used to automatically measure and store the corresponding position of the pointing device and/or marker.

According to a second aspect, the invention relates to a localization system for determining the spatial coordinates of at least one marker, comprising the following components:
a) A tracking device, i.e. a device that can localize some target unit in space.
b) A pointing device of the kind described above for indicating the spatial position of the marker to the tracking device. The pointing device therefore comprises a tip that can be located by the tracking device and that can be brought into a definite interaction with the marker as well as a sensor for detecting if said definite interaction takes place. Typically the target unit that is actually localized by the tracking device is the tip itself or at least a position on the same solid body as the tip.

According to its third aspect, the invention relates to an investigation apparatus, particularly a medical investigation apparatus, comprising the following components:
a) An imaging system for generating a (typically two- or three-dimensional) image of a region of interest that comprises at least one marker. The region of interest may for example be the body of a patient who has a plurality of markers attached to the skin.
b) A localization system of the kind described above for determining the spatial coordinates of the at least one marker. The localization system therefore comprises a tracking device and a pointing device with a tip that can be located by the tracking device and that can be brought into a definite interaction with the marker and with a sensor for detecting if said definite interaction takes place.
c) A data processing system, e.g. a microcomputer, for determining the image coordinates of the marker in the image that is generated by imaging system and for registering these image coordinates with the spatial coordinates determined by the localization system. As usual, the term “registration” shall denote in this context the determination of the geometrical transformation that maps image coordinates onto spatial coordinates and/or vice versa. Such a registration is needed for example during a medical intervention to find in a pre-operatively generated image those positions that correspond to the position of an instrument (e.g. a catheter tip) as actually measured by the localization system.

The localization system and the investigation apparatus according to the second and third aspect of the invention both comprise as a crucial component the pointing device according to the first aspect of the invention. Reference is therefore made to the description of this pointing device for more details on the localization system and the investigation apparatus.

The imaging system that is a part of the investigation apparatus may preferably comprise an X-ray device like an X-ray projection apparatus or a CT (Computed Tomography) scanner. Moreover, the imaging system may comprise a PET (Positron Emission Tomography) or SPECT (Single Photon Emission Computed Tomography) device, a Magnetic Resonance Imaging (MRI) device, or an ultrasound (US) device.

The localization system that is used in combination with the pointing device may operate based on any suitable principle for this purpose, e.g. based on magnetic, electromagnetic, optical or acoustical measurements. It may use “passive” or “active” target units, wherein the latter actively generate data or signals that allow to determine their spatial position and/or orientation. The localization system may for example generate an external (spatially or temporally inhomogeneous) magnetic field, wherein the corresponding (active) target unit is a magnetic field sensor that can measure magnitude and orientation of this field and wherein said measurements allow to infer the spatial position of the target unit with respect to the generator of the magnetic field. In another embodiment, the target unit may be a source of electromagnetic and/or acoustical radiation, e.g. of near infra red (NIR) or ultrasound, wherein the position of this source can be determined by the localization system via stereoscopic methods from the intersection of at least two independent lines of sight.

The definite interaction between the tip of the pointing device and the marker, which is detected by the sensor, may comprise the adoption of a predetermined relative positioning between the marker and the tip. This predetermined positioning may particularly correspond to a definite distance between the marker and the tip (or, more precisely, between dedicated points on the marker and the tip). A distance of zero would correspond in this context to a contact between marker and tip, while a distance larger than zero would correspond to a contactless measurement.

In an embodiment of the pointing device that is particularly suited for the aforementioned case, the pointing device comprises a proximity sensor and/or a contact sensor. These sensors can determine if a certain relative positioning between marker and tip prevails.

In another variant of the invention, the “definite interaction” comprises the exertion of a predetermined force or pressure on the marker by the tip of the pointing device (wherein the quantities “force” and “pressure” are here practically equivalent to each other, as force and pressure are related to each other by the associated area of force application). In this case it can be guaranteed that there is a sufficient contact between tip and marker which does however not exceed a given threshold in order to avoid an unintentional shift of the marker.

In an embodiment that is particularly in line with the aforementioned approach, the pointing device comprises a force sensor, e.g. a piezoelectric force sensor (as before, the term “force sensor” is used here synonymously to “pressure sensor”).

In a further development of the invention, the pointing device comprises an indicator for indicating that the definite interaction between tip and marker takes place. Said indicator may particularly be an optical indicator, for example an LED, or an acoustic indicator like a beeper. Thus a direct feedback can be given to the user to indicate the correct and/or false positioning of the pointing device.

Another variant of the invention is characterized by a readout-unit for triggering the automatic determination (and typically also storage) of the spatial coordinates of the pointing device when said device detects the occurrence of the definite interaction between its tip and a marker. The readout-unit may be located in the pointing device, in the tracking device, in the data processing system, or distributed over several components. It may for example comprise some indicator of the aforementioned kind in the pointing device that (e.g. electrically) signals if the required relative interaction between tip and marker prevails. Moreover, the “spatial coordinates of the pointing device” shall represent coordinates (e.g. of the tip) that finally allow to determine the spatial coordinates of the marker one is actually interested in.

### BRIEF DESCRIPTION OF THE DRAWINGS

These and other aspects of the invention will be apparent from and elucidated with reference to the embodiment(s) described hereinafter. These embodiments will be described by way of example with the help of the accompanying drawings in which:

FIG. 1 shows schematically an investigation apparatus according to the present invention;

FIG. 2 shows schematically a pointing device according to the present invention after first contact to a marker;

FIG. 3 shows the pointing device of FIG. 2 when it exerts more than a predetermined pressure on the marker.

Like reference numbers in the Figures refer to identical or similar components.

### DETAILED DESCRIPTION OF EMBODIMENTS

The present invention will be described in the following with respect to a medical application, though it is not limited to this area but can advantageously be applied in many different environments.

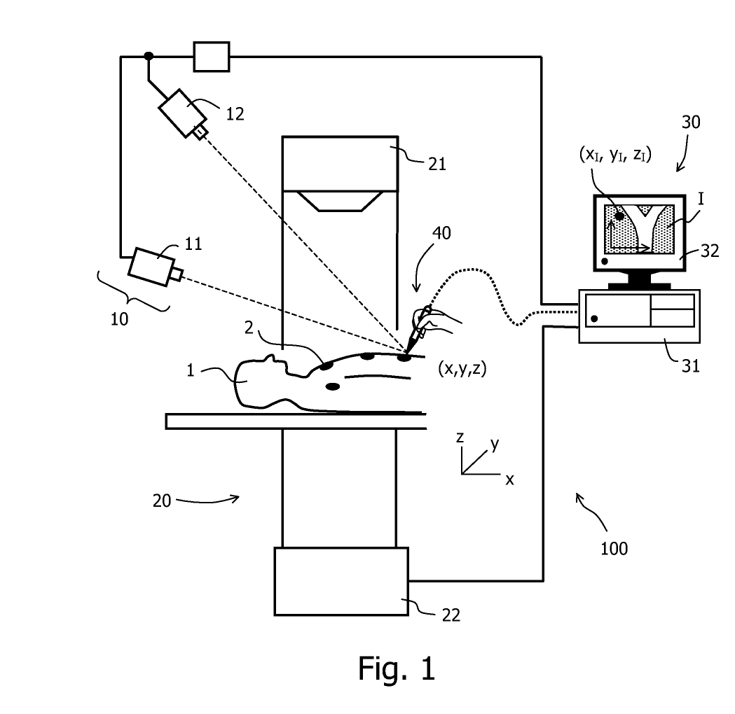

FIG. 1 shows schematically the setup of a medical intervention like a minimally invasive surgery. A set of markers 2 is attached to the skin of a patient 1 near a region of interest, e.g. the chest of the patient. The markers 2 may for example comprise radio-opaque bodies with a typical diameter of a few millimeters.

A first component of the investigation apparatus 100 according to the invention is an imaging system, in this case realized by a CT-scanner 20 with an X-ray source 21 and an X-ray detector 22 rotatably mounted on a gantry. The CT scanner 20 is coupled to a data processing device 30, e.g. a workstation 31 with a monitor 32, that controls the scanner and receives and processes the generated images. The CT scanner 20 usually generates two-dimensional X-ray projections from different directions, which can be used as such or which can be further processed to reconstruct slice images or three-dimensional images of the patient. At least one such two- or three-dimensional image I of the region of interest is generated pre-operatively and stored in the workstation 31 for later use during the medical intervention. The position of the markers 2 can be determined in this image I automatically or semi-automatically in image coordinates x_{I}, y_{I}, z_{I}.

The examination apparatus 100 further comprises a tracking device 10 for determining the spatial coordinates x, y, z of associated target units. The tracking device 10 is illustrated in the Figure by an optical system comprising at least two cameras 11, 12 which can be used to determine the position of an object in space according to the principles of stereoscopy, i.e. by calculating the intersection of two different lines of sight. The measurements of the tracking device 10 (raw data or processed data) are communicated to the workstation 31.

Other suitable tracking devices might for example use magnetic fields to determine the position of target units in space. They may comprise field coils for generating an inhomogeneous magnetic field within a region and (small) probe-coils for sensing the magnitude and/or orientation of this field at a particular point of interest, from which the coordinates of this point can be inferred.

Finally, the investigation apparatus 100 comprises a pointing device 40 which is used to manually indicate the positions of the markers 2 to the tracking device 10. In particular, the pointing device 40 carries some target unit which can be localized by the tracking device 10, e.g. a set of LEDs (not shown) that can be detected on recorded video images. Bringing this target unit to the markers 20 therefore allows to determine the spatial coordinates x, y, z of the markers.

When the spatial coordinates x, y, z of all markers 2 are known, the workstation 31 can register them with the corresponding image coordinates x_{I}, y_{I}, z_{I}. Once this registration is completed, it can be used to determine the x_{I},y_{I},z_{I}-image coordinates of any object that is localized by the tracking device 10 in x,y,z-coordinates. A typical object is for example an interventional device like the tip of a catheter, needle, or similar instrument.

The continuous visualization of the position of an interventional device on a pre-interventional acquired image is a powerful technique, which supports the physician with valuable information during the procedure and prevents the necessity for continuous imaging, e.g. using ionizing radiation exposure. Instead, the position and orientation of the interventional device are measured by an e.g. optical or electromagnetic tracking device 10 and continuously overlaid to a registered medical image I of the region of interest. The registration can be accomplished if the transformation from patient space to image space is known. As described above, this transformation can be determined by placing fiducial markers 2 on the skin of the patient 1, acquiring a diagnostic image I using an arbitrary imaging modality (MR, X-ray, CT, . . . ), determining the position x_{I},y_{I},z_{I} of the markers 2 in the image I, measuring the corresponding position x,y,z of the markers in patient space using an appropriate probe 40 in combination with the tracking device 10, and deriving the transformation (e.g. an affine transformation).

The accuracy of the forthcoming visualization of an interventional device in the image I strongly depends on the accuracy of the determined transformation. However, since the skin which carries the fiducial markers 2 is elastic, the measurements of the marker positions depend on the force with which the probe 40 is pushed towards the marker. The usually clumsy probe makes it difficult for a user to estimate the strength of the applied force, especially at markers which are partially hidden or difficult to approach. The resulting deformation of the skin can be in the order of several millimeters and limit the accuracy of the determined transformation significantly.

To address the aforementioned problems, a pointing device 40 is proposed here that comprises some sensor which indicates if a definite, predetermined interaction between marker and pointing device takes place. This definite interaction may both be a “good”, desired one as well as a “bad” one that should be avoided. Optionally the sensor may detect both “good” and “bad” interactions.

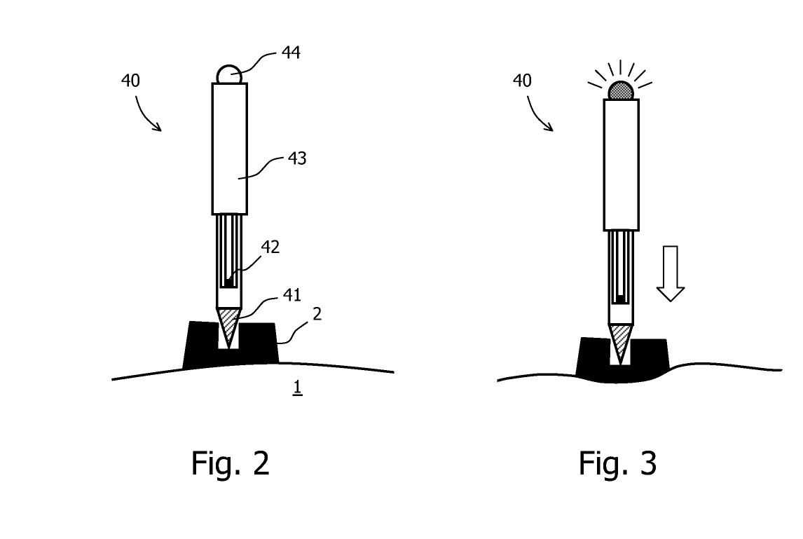

FIGS. 2 and 3 show a particular embodiment of such a pointing device 40. The generally pencil-shaped pointing device 40 comprises a handle 43 at which it can be held by a user and a tip 41 which has to be brought into contact with a marker 2. The position and optionally also orientation of the tip 41 can be determined by the tracking device 10 either directly or indirectly (by localizing a target unit at some other point on the pointing device 40).

The pointing device 40 further features a force sensor, e.g. a piezoelectric resistance 42, which is located such that it detects forces which are applied to the tip 41 of the instrument and which act relative to the handle 43 of the instrument. In case that the force along the main axis of the device exceeds a specified value, an optical or acoustic indicator is switched on, e.g. a light emitting diode (LED) 44. This situation is shown in FIG. 3, which also illustrates (exaggeratedly) the beginning deformation of the patient's skin by the applied force. If the predetermined threshold that triggers the activation of the LED 44 is sufficiently low, the force sensor 42 can practically act as a contact sensor that gives a notice as soon as its tip touches a marker. Optionally, the sensor may differentiate between “good” forces F in an allowable range (e.g. Fₘᵢₙ≦F≦Fₘₐₓ) and “bad” forces in a forbidden range (e.g. F>Fₘₐₓ), and indicate this to a user (e.g. via the activation of green or red LEDs, via a continuous or intermittent signal etc.). Optionally, the indication of a “good” force can be used to initiate an automatic readout of the measurement and store the measured position for registration. A dotted line in the Figure indicates a wired coupling between the pointing device 40 and a readout-unit in the workstation 31 for this purpose (a wireless coupling is of course possible, too).

The pointing device 40 is used during the determination of the transformation from patient-space to image-space by pointing it to the fiducial markers 2 fixed on the skin of the patient 1. As soon as the operator applies too much pressure, i.e. as soon as the skin starts to be deformed, the optical or acoustic signal indicates that the registration will fail and the measurement has to be repeated. This enables a significantly faster and much more accurate determination of the coordinate transformation. Therefore, the accuracy for the subsequently performed overlay of the position and orientation of an interventional device on an image of the patient is much higher. Moreover, a time-consuming repetition of the transformation determination can be prevented by ensuring that all markers are approached without deforming the skin including the marker position.

The proposed pointing device can particularly be applied to all clinical procedures which require an accurate registration from patient to image coordinate space and make use of optical or electromagnetic tracking systems and fiducial markers.

Finally it is pointed out that in the present application the term “comprising” does not exclude other elements or steps, that “a” or “an” does not exclude a plurality, and that a single processor or other unit may fulfill the functions of several means. The invention resides in each and every novel characteristic feature and each and every combination of characteristic features. Moreover, reference signs in the claims shall not be construed as limiting their scope.

1-11. (canceled)

12. A pointing device (40) for indicating the spatial position of a marker (2) to a tracking device (10), the pointing device (40) comprising:
a) a tip (41) that can be located by the tracking device (10);
b) a sensor (42) for detecting the occurrence of a definite interaction between said tip and the marker (2).

13. The pointing device (40) of claim 12, wherein the definite interaction comprises the adoption of a predetermined relative positioning between marker (2) and tip (41) of the pointing device (40).

14. The pointing device (40) of claim 12, wherein the pointing device (40) comprises a proximity sensor, a contact sensor or a force sensor.

15. The pointing device (40) of claim 14, wherein the pointing device (40) comprises a piezoelectric force sensor.

16. The pointing device (40) of claim 12, wherein the definite interaction comprises the exertion of a predetermined force or pressure on the marker (2).

17. The pointing device (40) of claim 12, wherein the pointing device (40) comprises an indicator (44) for indicating that the definite interaction takes place.

18. The pointing device (40) of claim 17, wherein the indicator (44) for indicating that the definite interaction takes place is an optical or an acoustic indicator.

19. The pointing device (40) of claim 12 comprising a readout-unit for triggering the determination of the spatial coordinates (x,y,z) of the pointing device (40) when said pointing device (40) detects the occurrence of the definite interaction.

20. A localization system for determining the spatial coordinates (x,y,z) of a marker (2), the localization system comprising
a) a tracking device (10); and
b) a pointing device (40) for indicating the spatial position of the marker (2) to the tracking device (10),
the pointing device (40) comprising a tip (41) capable of being located by the tracking device (10) and a sensor (42) for detecting the occurrence of a definite interaction between said tip and the marker (2).

21. The localization system of claim 20, wherein the tracking device (10) operates based on magnetic, electromagnetic, optical or acoustical measurements.

22. The localization system (10) of claim 19, wherein the definite interaction comprises the adoption of a predetermined relative positioning between marker (2) and tip (41) of the pointing device (40).

23. The localization system (10) of claim 20, wherein the pointing device (40) comprises an indicator (44) for indicating that the definite interaction takes place.

24. The localization system (10) of claim 20, comprising a readout-unit for triggering the determination of the spatial coordinates (x,y,z) of the pointing device (40) when said pointing device (40) detects the occurrence of the definite interaction.

25. An investigation apparatus, comprising:
a) an imaging system (20) for generating an image (1) of a region of interest comprising at least one marker (2);
b) a localization system (10) for determining the spatial coordinates (x,y,z) of the marker (2); and
c) a data processing system (30) for determining the image coordinates (x_{I},y_{I},z_{I}) of the marker (2) in the image (I) and for registering them with the spatial coordinates (x,y,z) determined by the localization system,
wherein the localization system includes a tracking device (10) and a pointing device (40) for indicating the spatial position of the marker (2) to the tracking device (10), the pointing device (40) comprising a tip (41) capable of being located by the tracking device (10) and a sensor (42) for detecting the occurrence of a definite interaction between said tip and the marker (2).

26. The investigation apparatus of claim 25, wherein the imaging system comprises an X-ray device, a CT-scanner (20), a PET, a SPECT, an MRI or an ultrasound device.

27. The investigation apparatus of claim 25, wherein the definite interaction comprises the adoption of a predetermined relative positioning between marker (2) and tip (41) of the pointing device (40).

28. The investigation apparatus of claim 25, wherein the pointing device (40) comprises at least one of a proximity sensor, a contact sensor or a force sensor.

29. The investigation apparatus of claim 25, wherein the definite interaction comprises the exertion of a predetermined force or pressure on the marker (2).

30. The investigation apparatus according to claim 25, wherein the pointing device (40) comprises an indicator (44) for indicating that the definite interaction takes place.

31. The investigation apparatus according to claim 25, wherein the investigation apparatus comprises a readout-unit for triggering the determination of the spatial coordinates (x,y,z) of the pointing device (40) when said pointing device (40) detects the occurrence of the definite interaction.
